(19) United States
(12) Patent Application Publication
(10) Pub. No.: US 2024/0002321 A1
(43) Pub. Date: Jan. 4, 2024

(54) HEAT RECOVERY FROM FLUE GAS DURING ALKYL TERT-BUTYL ETHER PRODUCTION

(71) Applicant: SABIC GLOBAL TECHNOLOGIES B.V., BERGEN OP ZOOM (NL)

(72) Inventors: Mohammed Bismillah ANSARI, Riyadh (SA); Vijay Dinkar BODAS, Riyadh (SA); Muhammad Imran YAQUB, Riyadh (SA)

(73) Assignee: SABIC GLOBAL TECHNOLOGIES B.V., BERGEN OP ZOOM (NL)

(21) Appl. No.: 18/253,998

(22) PCT Filed: Nov. 23, 2021
(86) PCT No.: PCT/IB2021/060897

§ 371 Date: May 23, 2023

(30) Foreign Application Priority Data
Nov. 24, 2020 (EP) 20209419.9

(51) Int. Cl. C07C 41/42

(52) U.S. Cl. CPC C07C 41/42

(57) ABSTRACT

Systems and methods for producing an alkyl tert-butyl ether are disclosed. The methods include providing heat to a reboiler of a distillation column of an alkyl tert-butyl ether production unit from a flue gas emanating from a unit carrying out a catalyst regeneration process.

### CROSS REFERENCE TO RELATED APPLICATIONS

This application claims the benefit of priority to European Patent Application No. 20209419.9, filed Nov. 24, 2020, the entire contents of which are hereby incorporated by reference in their entirety.

### FIELD OF INVENTION

The present invention generally relates to optimization of heat integration for endothermic processes. More specifically, the present invention relates to a process of recovering heat from one or more catalyst regeneration processes to provide reaction heat for an alkyl tert-butyl ether production process.

### BACKGROUND OF THE INVENTION

Heat integration and optimization are imperative in the chemical industry for improving energy efficiency and lowering production costs. Generally, at least a portion of heat needed by endothermic chemical reactions and/or processes can be provided by other exothermic chemical production processes, such that the need for heat via directly burning of fuel is mitigated.

Methyl tert-butyl ether (MTBE), commonly used as a gasoline blending component, can be synthesized via an etherification reaction between isobutylene and methanol. In the MTBE production process multiple steps require heating. Isobutylene feed is produced via dehydrogenation of isobutane, which is an endothermic process. The etherification reaction of isobutylene and methanol is carried out at 60 to 90° C., which requires heating to maintain the reaction temperature. Furthermore, heating is also needed for separating MTBE from an effluent stream in MTBE synthesis reactors via distillation to produce the MTBE product stream. Thus, the MTBE production process is energy intensive. Currently, although some heating network optimization has been done for the conventional MTBE production process, the energy consumption for this process remains high.

Overall, while systems and methods for providing heat for MTBE production exist, the need for improvements in this field persists in light of at least the aforementioned drawback for the conventional systems and methods.

### BRIEF SUMMARY OF THE INVENTION

A solution to at least the above mentioned problem associated with the systems and methods for providing heat to the MTBE production process is discovered. The solution resides in a system and a method for producing an alkyl tert-butyl ether that includes providing heat to a reboiler of a separation column or a reboiler of a reactive distillation column of an alkyl tert-butyl ether production unit using a flue gas emanating from a unit carrying out a catalyst regeneration process. This can be beneficial for at least recovering some heat from a waste gas stream to reduce energy consumption, thereby reducing the production cost for the alkyl tert-butyl ether. Furthermore, the unit for carrying out the catalyst regeneration process can include an isobutane dehydrogenation unit configured to produce isobutylene as a feed for an MTBE synthesis reactor, further reducing energy consumption for MTBE production. Moreover, at least some heat from the flue gas from regenerating isobutane dehydrogenation catalyst can be recovered to produce superheated steam, which can be used for providing heat for other processes. Therefore, the systems and methods of the present invention provide a technical solution to the problem associated with the conventional systems and methods for producing an alkyl tert-alkyl ether.

Embodiments of the invention include a method of producing an alkyl tert-butyl ether. The method comprises providing heat to a reboiler of a distillation column of an alkyl tert-butyl ether production unit from a flue gas emanating from a unit carrying out a catalyst regeneration process.

Embodiments of the invention include a method of producing methyl tert-butyl ether (MTBE). The method comprises providing heat to a reboiler of an MTBE purification column and/or reboiler of a reactive distillation column of an MTBE production unit from a flue gas emanating from an isobutane dehydrogenation unit carrying out the catalyst regeneration process.

Embodiments of the invention include a method of producing methyl tert-butyl ether (MTBE). The method comprises flowing a flue gas stream generated by regenerating a catalyst of a dehydrogenation unit into an air waste heat boiler. The method includes heating steam, in the air waste heat boiler, by the flue gas stream to produce a cooled flue gas stream. The method includes flowing at least a portion of the cooled flue gas stream into a reboiler of an MTBE purification column or a reboiler of a reactive distillation column of an MTBE production unit. The method further includes providing heat to the reboiler by using the cooled flue gas stream as a heating medium.

The following includes definitions of various terms and phrases used throughout this specification.

The terms “about” or “approximately” are defined as being close to as understood by one of ordinary skill in the art. In one non-limiting embodiment the terms are defined to be within 10%, preferably, within 5%, more preferably, within 1%, and most preferably, within 0.5%.

The terms “wt. %”, “vol. %” or “mol. %” refer to a weight, volume, or molar percentage of a component, respectively, based on the total weight, the total volume, or the total moles of material that includes the component. In a non-limiting example, 10 moles of component in 100 moles of the material is 10 mol. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, include any measurable decrease or complete inhibition to achieve a desired result.

The term “effective,” as that term is used in the specification and/or claims, means adequate to accomplish a desired, expected, or intended result.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The term “NOX,” as that term is used in the specification and/or claims, means nitrogen oxides including nitrogen dioxide and/or nitric oxide.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process of the present invention can “comprise,” “consist essentially of,” or “consist of” particular ingredients, components, compositions, etc., disclosed throughout the specification.

The term “primarily,” as that term is used in the specification and/or claims, means greater than any of 50 wt. %, 50 mol. %, and 50 vol. %. For example, “primarily” may include 50.1 wt. % to 100 wt. % and all values and ranges there between, 50.1 mol. % to 100 mol. % and all values and ranges there between, or 50.1 vol. % to 100 vol. % and all values and ranges there between.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

For a more complete understanding, reference is now made to the following descriptions taken in conjunction with the accompanying drawings, in which:

FIGS. 1A and 1B show systems for recovering heat from a flue gas stream to a reboiler of a distillation column of an alkyl tert-butyl ether production system, according to embodiments of the invention; FIG. 1A shows a system for recovering heat from a flue gas stream to a reboiler of a non-reactive distillation column; FIG. 1B shows a system for recovering heat from a flue gas stream to a reboiler of a reactive distillation column; FIG. 1C shows a system according to embodiments of the invention having two gas turbines configured to supply turbine exhaust gas stream to catalytic reactor as a regeneration gas; FIG. 1D discloses a system according to embodiments of the invention which has one gas turbine wherein an exhaust gas stream from the gas turbine is fed directly to an air heater without the use of a process air compressor.

FIG. 2 shows a schematic flowchart of a method for producing an alkyl tert-butyl ether, according to embodiments of the invention.

### DETAILED DESCRIPTION OF THE INVENTION

Currently, alkyl tert-butyl ethers (e.g., MTBE) are produced via multiple energy intensive steps. Thus, the energy cost and consequently overall production cost for producing alkyl tert-butyl ethers are high. The present invention provides a solution to this problem. The solution is premised on recovering heat from a flue gas from a catalyst regeneration process and providing the recovered heat to a reboiler of a distillation column (a non-reactive or a reactive distillation column) of an alkyl tert-butyl ether production process, thereby improving energy efficiency. Additionally, the flue gas can be obtained from an isobutane dehydrogenation reactor, which is configured to produce an isobutylene feed stream for producing the alkyl tert-butyl ether, thus, further optimizing heat integration in the alkyl tert-butyl ether production process. Further still, at least a portion of the heat of the flue gas can be used to super heat steam, which can be used to provide heat for other steps of the alkyl tert-butyl ether production process to further improve energy efficiency. These and other non-limiting aspects of the present invention are discussed in further detail in the following sections.

### A. System for Recovering Heat for Alkyl Tert-Butyl Ether Production

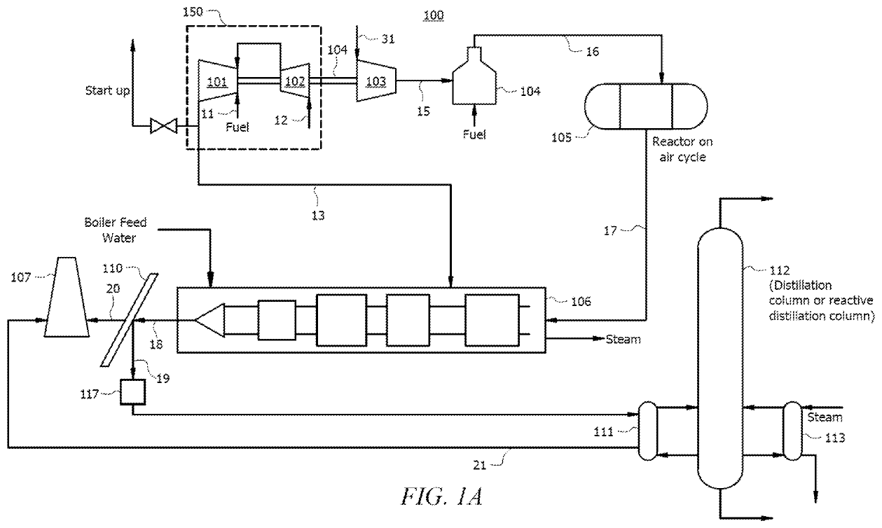

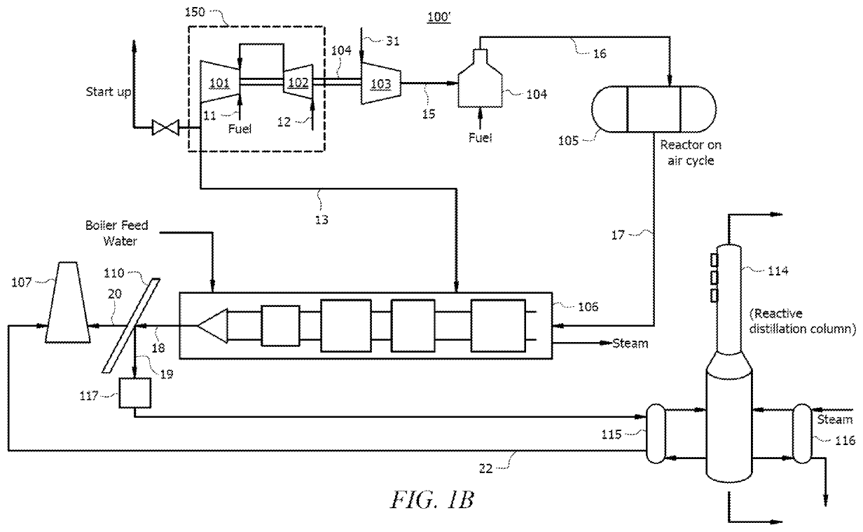

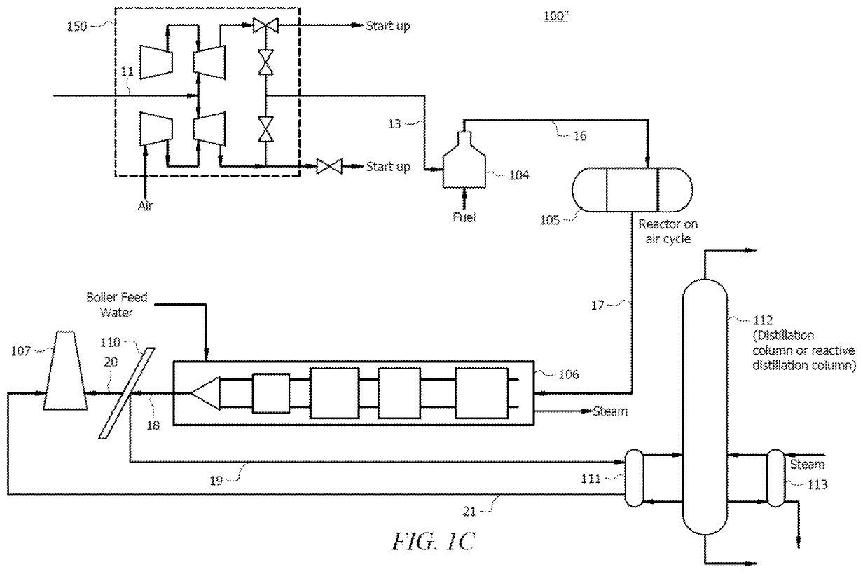

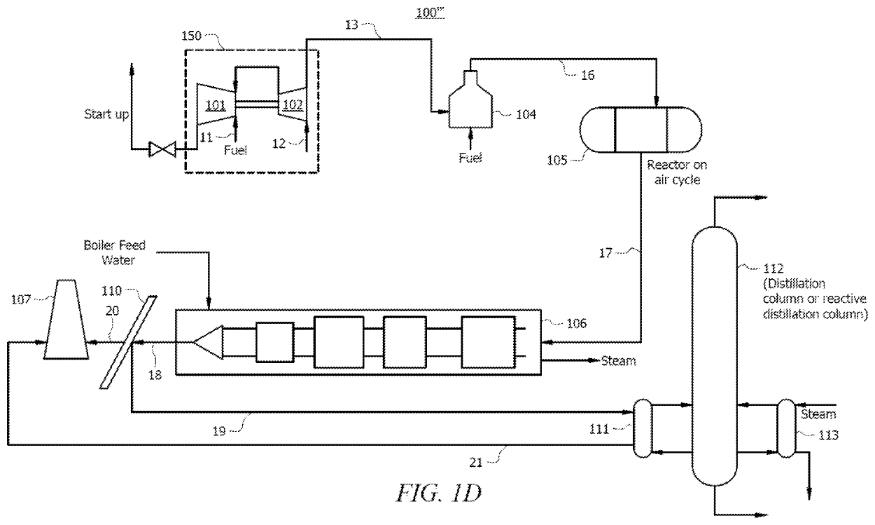

In embodiments of the invention, the system for recovering heat from a flue gas to an alkyl tert-butyl ether production unit includes a gas turbine, a dehydrogenation unit, an air waste heat boiler, and a distillation column (including a non-reactive distillation column or a reactive distillation column). Notably, the system is capable of reducing energy consumption and increasing efficiency for producing an alkyl tert-butyl ether compared to conventional systems. With reference to FIG. 1A, a schematic diagram is shown for system 100, which is used for recovering heat from a flue gas stream and providing the recovered heat to an alkyl tert-butyl ether production process.

According to embodiments of the invention, system 100 includes gas turbine unit 150 (combination of 101 and 102) configured to combust a fuel of first fuel stream 11 in first stream 12 comprising an oxidant to produce turbine exhaust gas stream 13. Gas turbine unit 150 is further configured to drive process air compressor 103 via shaft 104. First stream 12 can include air. The air of first stream 12 may be under ambient conditions. In embodiments of the invention, the fuel of first fuel stream 11 includes natural gas, hydrogen, methane, ethane, carbon monoxide, carbon dioxide, or combinations thereof. The hydrogen of first fuel stream 11 may be produced and recovered from a hydrocarbon dehydrogenation process.

In embodiments of the invention, process air compressor 103 can be an air compressor of a hydrocarbon dehydrogenation unit. The dehydrogenation unit can include an n-butane dehydrogenation unit, an isobutane dehydrogenation unit, a propane dehydrogenation unit, an isopentane dehydrogenation unit, a propane dehydrogenation unit, or combinations thereof. Process air compressor 103 is configured to compress inlet gas stream 31 to form high pressure stream 15. Inlet gas stream 31 may include an air stream. Inlet gas stream 31 may be a hot gas stream from a waste air vent of an MTBE production unit. According to embodiments of the invention, the hot gas stream from a waste air vent of an MTBE production unit comprises oxygen, nitrogen, carbon dioxide, carbon monoxide, oxides of sulfur and/or nitrogen, or combinations thereof. High pressure stream 15 can include atmospheric air (having 79% nitrogen and 21% oxygen on dry, CO₂ and argon-free basis with traces of CO₂ (about 330-450 ppm) and argon (0.93%) and water vapor in accordance with local humidity conditions compressed to a pressure of about 2.2 to 3 bar (abs) and all ranges and values there between.

According to embodiments of the invention, an outlet of process air compressor 103 is in fluid communication with an inlet of air heater 104 such that high pressure stream 15 flows from process air compressor 103 to air heater 104. Air heater 104 may be configured to combust a fuel and high pressure stream 15 to produce regeneration gas stream 16. In embodiments of the invention, regeneration gas stream 16 is at a temperature of 600 to 730° C. and all ranges and values there between including ranges of 600 to 610° C., 610 to 620° C., 620 to 630° C., 630 to 640° C., 640 to 650° C., 650 to 660° C., 660 to 670° C., 670 to 680° C., 680 to 690° C., 690 to 700° C., 700 to 710° C., 710 to 720° C., and 720 to 730° C. In embodiments of the invention, regeneration gas stream 16 includes 1 to 15 vol. % oxygen gas, 74 to 79 vol. % nitrogen gas, 2 to 4 vol. % CO₂, 5 to 8 vol. % water vapor, and a minor amount of argon.

In embodiments of the invention, an outlet of air heater 104 is in fluid communication with an inlet of catalytic reactor 105 such that regeneration gas stream 16 flows from air heater 104 to catalytic reactor 105. Catalytic reactor 105 comprises a catalyst disposed therein. Catalytic reactor 105, in embodiments of the invention, can include a dehydrogenation reactor configured to catalytically dehydrogenate a hydrocarbon to produce one or more unsaturated hydrocarbons. The dehydrogenation reactor can include an n-butane dehydrogenation reactor, an isobutane dehydrogenation reactor, a propane dehydrogenation reactor, and/or an isopentane dehydrogenation reactor.

In embodiments of the invention, catalytic reactor 105 is in regeneration mode and regeneration gas stream 16 is configured to regenerate spent catalyst of catalytic reactor 105 to produce regenerated catalyst and flue gas stream 17. In embodiments of the invention, flue gas stream 17 is at a temperature in a range of 530 to 560° C. Flue gas stream 17 may include 1 to 15 vol. % oxygen.

According to embodiments of the invention, an outlet of catalytic reactor 105 is in fluid communication with air waste heat boiler and NOX removal unit 106 such that flue gas stream 17 flows from catalytic reactor 105 to air waste heat boiler and NOX removal unit 106. In embodiments of the invention, air waste heat boiler and NOX removal unit 106 is configured to heat steam by using at least a portion of flue gas stream 17 and/or at least a portion of turbine exhaust gas stream 13 as a heating medium to produce superheated steam, and/or remove nitrogen oxides from flue gas stream 17 to produce cooled flue gas stream 18. In embodiments of the invention, air waste heat boiler and NOX removal unit 106 comprises a steam superheater, a boiler, and an economizer. Air waste heat boiler and NOX removal unit 106 may further comprise a selective catalytic NO_{X} removal system for removing nitrogen oxides.

As an alternative to, or in addition to using at least a portion of turbine exhaust gas stream 13 as a heating medium for air waste heat boiler and NOX removal unit 106, at least a portion of turbine exhaust gas stream 13 may be flowed into catalytic reactor 105 as regeneration gas for regenerating the catalyst therein. In embodiments of the invention, gas turbine unit 150 can include two gas turbines operated in parallel. The two gas turbines can be configured to supply turbine exhaust gas stream 13 to catalytic reactor 105 as a regeneration gas (as shown in system 100″ of FIG. 1C). Exhaust gas stream 13 from one or more gas turbines of gas turbine unit 150 can be heated in air heater 104 and the heated exhaust gas stream can be flowed into catalytic reactor 105 as the regeneration gas. In embodiments of the invention, as shown in system 100′″ of FIG. 1D, gas turbine unit 150 includes one gas turbine, as exhaust gas stream 13 from the gas turbine is fed directly to air heater 104 without the use of process air compressor 103.

According to embodiments of the invention, a tapping device 110 may be installed between an outlet of air waste heat boiler and NOX removal unit 106 and an inlet of an air waste heat boiler stack 107. Tapping device 110, in embodiments of the invention, is configured to divide cooled flue gas stream 18 to form recovered flue gas stream 19 and vented flue gas stream 20. Tapping device 110 may include a valve, a baffle plate, a damper, or combinations thereof. According to embodiments of the invention, an outlet of air waste heat boiler and NOX removal unit 106 is in fluid communication with an inlet of air waste heat boiler stack 107 such that vented flue gas stream 20 flows from air waste heat boiler and NOX removal unit 106 to air waste heat boiler stack 107. In embodiments of the invention, process air compressor 103, air heater 104, catalytic reactor 105, air waste heat boiler and NOX removal unit 106, and/or air waste heat boiler stack 107 may be part of a hydrocarbon dehydrogenation unit.

According to embodiments of the invention, an outlet of tapping device 110 is in fluid communication with reboiler 111 such that recovered flue gas stream 19 flows from tapping device 110 to reboiler 111. In embodiments of the invention, reboiler 111 can include a flue gas driven reboiler. Reboiler 111 may be a reboiler of a non-reactive distillation column 112. Non-reactive distillation column 112 can be configured to separate alkyl tert-butyl ether (e.g., MTBE and ETBE) from an effluent stream of an alkyl tert-butyl ether (e.g., MTBE and ETBE) to form an alkyl tert-butyl ether product stream. Non-reactive distillation column 112 can include two or more reboilers including reboiler 111 and a steam driven reboiler 113. In embodiments of the invention, non-reactive distillation column 112 is part of an alkyl tert-butyl ether production system that includes a primary alkyl tert-butyl ether synthesis reactor and a secondary alkyl tert-butyl ether synthesis reactor in series. According to embodiments of the invention, reboiler 111 is configured to utilize recovered flue gas stream 19 as a heating medium to heat liquid content therein and produce exhaust flue gas stream 21. In embodiments of the invention, an outlet of reboiler 111 is in fluid communication with an inlet of air waste heat boiler stack 107 such that exhaust flue gas stream 21 flows from reboiler 111 to air waste heat boiler stack 107.

As shown in FIG. 1B, according to embodiments of the invention, system 100′ includes all the units and streams as system 100 shown in FIG. 1A except that, in system 100′, an outlet of tapping device 110 is in fluid communication with second reboiler 115 of reactive distillation column 114 such that recovered flue gas stream 19 flows from tapping device 110 to second reboiler 115. Reactive distillation column 114 may be part of an alkyl tert-butyl ether production system that includes a primary alkyl tert-butyl ether synthesis reactor and reactive distillation column 114 in series. Reactive distillation column 114 can comprise two or more reboilers including second reboiler 115 and second steam driven reboiler 116. Second reboiler 115 may be a flue gas driven reboiler configured to utilize recovered flue gas stream 19 as a heating medium to heat content therein and produce second exhaust flue gas stream 22. An outlet of second reboiler 115 can be in fluid communication with an inlet of waste heat boiler stack 107 such that second exhaust flue gas stream 22 flows from second reboiler 115 to air waste heat boiler stack 107.

### B. Method of Producing Alkyl Tert-Butyl Ether

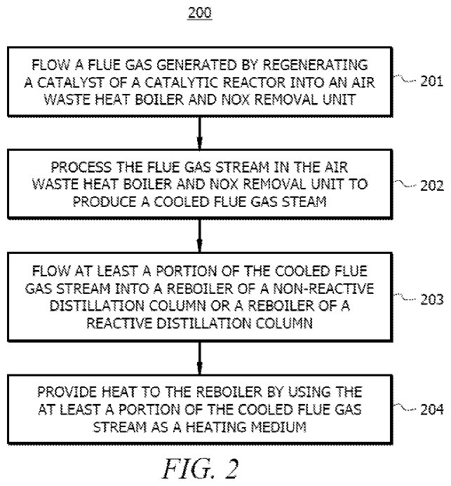

Methods of producing an alkyl tert-butyl ether, including MTBE and/or ETBE, have been discovered. As shown in FIG. 2, embodiments of the invention include method 200 for producing heat for an alkyl tert-butyl ether production process with improved energy efficiency and reduced production cost compared to conventional methods. Method 200 may be implemented by system 100 or system 100′, as shown in FIG. 1A or FIG. 1B, respectively, and described above.

According to embodiments of the invention, as shown in block 201, method 200 includes flowing flue gas stream 17 generated by regenerating a catalyst of catalytic reactor 105 into air waste heat boiler and NOX removal unit 106. In embodiments of the invention, catalytic reactor 105 includes a dehydrogenation reactor of a dehydrogenation unit. In embodiments of the invention, catalytic reactor 105 includes an isobutane dehydrogenation reactor. The catalyst of catalytic reactor 105 can include chromium on alumina or platinum on alumina. Flue gas stream 17 may be produced by utilizing first regeneration gas stream 13, high pressure stream 15, or regeneration gas stream 16 to regenerate the catalyst of catalytic reactor 105. In embodiments of the invention, flue gas stream 17 is at a temperature of 540 to 640° C. and all ranges and values there between including ranges of 540 to 550° C., 550 to 560° C., 560 to 570° C., 570 to 580° C., 580 to 590° C., 590 to 600° C., 600 to 610° C., 610 to 620° C., 620 to 630° C., 630 to 640° C., and 640 to 650° C. Flue gas stream 17 may include 1 to 15 mol. % oxygen gas, 70 to 77 mol. % nitrogen gas, 4 to 6 mol. % CO₂ gas, and 2 to 8 mol. % water vapor.

According to embodiments of the invention, as shown in block 202, method 200 includes processing flue gas stream 17 in air waste heat boiler and NOX removal unit 106 to produce cooled flue gas stream 18. In embodiments of the invention, processing at block 202 includes heating steam in the air waste heat boiler section of air waste heat boiler and NOX removal unit 106, by flue gas stream 17 to produce superheated steam. Processing at block 202 further includes removing nitrogen oxides from flue gas stream 17 by the NOX removal section of air waste heat boiler and NOX removal unit 106. In embodiments of the invention, cooled flue gas stream 18 is at a temperature of 210 to 230° C. and all ranges and values there between including ranges of 210 to 212° C., 212 to 214° C., 214 to 216° C., 216 to 218° C., 218 to 220° C., 220 to 222° C., 222 to 224° C., 224 to 226° C., 226 to 228° C., and 228 to 230° C. Cooled flue gas stream 18 may include nitrogen oxides less than 86 nanogram/MMBtu for its gas fired system component and 130 nanogram/MMBtu for its oil fired system fraction, and NOₓ=0.0150 (14.4)/Y+F, percent by volume calculated at 15% oxygen on dry basis, where Y is manufacturers or actual peak load not exceeding 14.4 KJ/watt hr and F is allowance of fuel nitrogen content in accordance with 40 CFR Ch. I (7-1-12 edition); for its gas turbine fraction.

According to embodiments of the invention, as shown in block 203, method 200 includes flowing at least a portion of cooled flue gas stream 18, including recovered flue gas stream 19, into reboiler 111 of non-reactive distillation column 112 or second reboiler 115 of reactive distillation column 114 of an alkyl tert-butyl ether production unit. The flowing at block 203 may be conducted by using a blower 117 to drive recovered flue gas stream 19 from tapping device 110 to reboiler 111 and/or second reboiler 115. In embodiments of the invention, the alkyl tert-butyl ether production unit is an MTBE production unit that includes (i) catalytic reactor 105 as an isobutane dehydrogenation unit configured to produce isobutylene, (ii) a primary MTBE synthesis reactor configured to react the isobutylene with methanol to produce MTBE, (iii) a secondary MTBE synthesis reactor configured to react unreacted isobutylene and methanol in an effluent of the primary MTBE synthesis reactor to produce additional MTBE, (iv) non-reactive distillation column 112 configured to separate MTBE from an effluent from secondary MTBE synthesis reactor to produce an MTBE product stream comprising primarily MTBE. Non-reactive distillation column 112 may include reboiler 111 and/or steam driven reboiler 113. In embodiments of the invention, non-reactive distillation column 112 is operated at a bottom temperature range of 135 to 145° C. and all ranges and values there between including ranges of 135 to 137° C., 137 to 139° C., 139 to 141° C., 141 to 143° C., and 143 to 145° C. Non-reactive distillation column 112 may be operated at an overhead temperature range of 50 to 55° C. and an operating pressure of 7.5 to 8 kgf/cm² (gauge).

In embodiments of the invention, the alkyl tert-butyl ether production unit is an MTBE production unit that includes (a) catalytic reactor 105 adapted to dehydrogenate isobutane to produce isobutylene, (b) an MTBE synthesis reactor configured to react the isobutylene with methanol to produce MTBE, (c) reactive distillation column 114 configured to react unreacted isobutylene and methanol in an effluent from the MTBE synthesis reactor to produce additional MTBE and separating reaction mixture therein to produce an MTBE product stream comprising primarily MTBE. Reactive distillation column 114 can include second reboiler 115 and/or second steam driven reboiler 116. Reactive distillation column 114 can include an etherification catalyst comprising sulfonic functionalized polystyrene divinyl benzene supported cation exchange resin, macro reticular, or combinations thereof. In embodiments of the invention, reactive distillation column 114 is operated at a bottom temperature range of 135 to 145° C. and all ranges and values there between including ranges of 135 to 137° C., 137 to 139° C., 139 to 141° C., 141 to 143° C., and 143 to 145° C. Reactive distillation column 114 may be operated at an overhead temperature range of 50 to 55° C. and an operating pressure of 7.5 to 8 kgf/cm² (gauge). In embodiments of the invention, at least a portion of cooled flue gas stream 18, including vented flue gas stream 20, is flowed to air waste heat boiler stack 107.

According to embodiments of the invention, as shown in block 204, method 200 includes providing heat to reboiler 111 and/or second reboiler 115 by using the at least a portion of cooled flue gas stream 18, including recovered flue gas stream 19, as a heating medium. In embodiments of the invention, at block 204, recovered flue gas stream 19 is cooled in reboiler 111 and/or second reboiler 115 to produce exhaust flue gas stream 21 and/or second exhaust flue gas stream 22, respectively. Exhaust flue gas stream 21 and/or second exhaust flue gas stream 22 may be flowed to air waste heat boiler stack 107. In embodiments of the invention, exhaust flue gas stream 21 is at a temperature of 155 to 170° C. and all ranges and values there between. Second exhaust flue gas stream 22 is at a temperature of 155 to 170° C. and all ranges and values there between.

Although embodiments of the present invention have been described with reference to blocks of FIG. 2, it should be appreciated that operation of the present invention is not limited to the particular blocks and/or the particular order of the blocks illustrated in FIG. 2. Accordingly, embodiments of the invention may provide functionality as described herein using various blocks in a sequence different than that of FIG. 2

The systems and processes described herein can also include various equipment that is not shown and is known to one of skill in the art of chemical processing. For example, some controllers, piping, computers, valves, pumps, heaters, thermocouples, pressure indicators, mixers, heat exchangers, and the like may not be shown.

As part of the disclosure of the present invention, specific examples are included below. The examples are for illustrative purposes only and are not intended to limit the invention. Those of ordinary skill in the art will readily recognize parameters that can be changed or modified to yield essentially the same results.

### Example

### Heat Recovery from Flue Gas Obtained by Regenerating Catalyst of a Dehydrogenation Unit

Both simulation and experiment were conducted for a heat recovery process from a flue gas stream obtained by regenerating a catalyst of a dehydrogenation unit. The flue gas stream was then flowed into a reboiler of a distillation column (non-reactive distillation column or a reactive distillation column) to provide heat for the reboiler. The regenerating gas stream used for regenerating the catalyst was produced by (A) an air compressor of a dehydrogenation process driven by an 80% loaded driver gas turbine at low (less than 0.05 kgf/cm² (gauge) back pressure in system 100′ as shown in FIG. 1B, (B) two parallel gas turbines directly exhausting to dehydrogenation reactors to produce regeneration air and operating at 75% load, each at high back pressure corresponding to dehydrogenation reactor pressure drop in system 100″ as shown in FIG. 1C, and (C) one gas turbine directly exhausting to dehydrogenation reactors to produce regeneration air and operating at 75% load at high back pressure corresponding to dehydrogenation reactor pressure drop in system 100′″ as shown in FIG. 1D. The results are shown in Table 1.

TABLE 1
Results for Heat Recovery from Flue Gas

| | | Exam- | Exam- | Exam- |
|---|---|---|---|---|
| Description | Units | ple 1. | ple 2 | ple 3 |
| Dehydrogenation process variant | | A | B | C |
| (Flue gas generation process) | | | | |
| Type of process (Refer to FIGS.) | | FIG. 1B | FIG. 1C | FIG. 1D |
| Fuel gas | btu/scf | 1080 | 1080 | 1080 |
| Flue gas | t/hr | 1040 | 680 | 340 |
| flue gas reboiler inlet | Deg. C. | 210 | 210 | 210 |
| (Before stack entrance) | | | | |
| Cp | cal/gm/ | 0.24 | 0.24 | 0.24 |
| | Deg. C. | | | |
| flue gas reboiler exit | Deg. C. | 170 | 170 | 170 |
| (After flue gas heat recovery) | | | | |
| Rec heat | MMKcal/ | 10.18 | 6.66 | 3.33 |
| | hr | | | |
| Rec heat | MMBtu/hr | 40.37 | 26.40 | 13.20 |
| Boiler Fuel firing saved (energy) | MMSCFD | 1.09 | 0.72 | 0.36 |

In the context of the present invention, at least the following 15 embodiments are disclosed. Embodiment 1 is a method of producing an alkyl tert-butyl ether. The method includes providing heat to a reboiler of a distillation column of an alkyl tert-butyl ether production unit from a flue gas emanating from a unit carrying out a catalyst regeneration process. Embodiment 1 is the method of embodiment 1, wherein the distillation column includes a non-reactive distillation column, and/or a reactive distillation column.

Embodiment 3 is a method of producing an alkyl tert-butyl ether. The method includes flowing a flue gas stream generated by regenerating a catalyst of a dehydrogenation unit into an air waste heat boiler. The method further includes processing the flue gas stream to produce a cooled flue gas stream. The method still further includes flowing at least a portion of the cooled flue gas stream into a reboiler of a non-reactive distillation column or a reboiler of a reactive distillation column of an alkyl tert-butyl ether production unit. The method also includes providing heat to the reboiler by using the cooled flue gas stream as a heating medium. Embodiment 4 is the method of embodiment 3, wherein the alkyl tert-butyl ether includes methyl tert-butyl ether (MTBE) and/or ethyl tert-butyl ether (ETBE). Embodiment 5 is the method of either of embodiments 3 or 4, wherein the unit carrying out the catalyst regeneration process includes an isobutane dehydrogenation unit. Embodiment 6 is the method of embodiment 5, wherein the isobutane dehydrogenation unit is configured to produce isobutylene as feedstock for MTBE or ETBE synthesis. Embodiment 7 is the method of any of embodiments 3 to 6, further including flowing at least a portion of the cooled flue gas stream into a stack for the air waste heat boiler. Embodiment 8 is the method of embodiment 7, wherein, by providing heat to the reboiler, the cooled flue gas is further cooled to form an exhaust flue gas flowed from the reboiler to the stack for the air waste heat boiler. Embodiment 9 is the method of any of embodiments 6 to 8, wherein a tapping device is installed between an outlet of the air waste heat boiler and the inlet of the stack for the air waste heat reboiler, for splitting at least a portion of cooled flue gas stream that is flowed into the reboiler. Embodiment 10 is the method of embodiment 9, wherein the tapping device includes a valve, a baffle plate, or a damper. Embodiment 11 is the method of any of embodiments 3 to 10, wherein the non-reactive distillation column and the reactive distillation column each include (1) a flue gas driven reboiler configured to use the cooled flue gas stream as a heating medium and (2) a steam driven reboiler configured to use steam as a heating medium. Embodiment 12 is the method of any of embodiments 3 to 11, wherein the cooled flue gas stream is flowed through the reboiler by a blower. Embodiment 13 is the method of any of embodiments 3 to 12, wherein the flue gas stream is at a temperature in a range of 540 to 640° C., and the cooled flue gas stream is at a temperature of 210 to 230° C. Embodiment 14 is the method of any of embodiments 3 to 13, wherein the flue gas stream contains 1 to 15 mol. % oxygen gas, 70 to 77 mol. % nitrogen gas, 4 to 6 mol. % CO₂ gas, 2 to 8 mol. % water vapor. Embodiment 15 is the method of any of embodiments 3 to 14, wherein the regenerating gas can include at least a portion of hot gas from a waste air vent of an MTBE production unit.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A method of producing an alkyl tert-butyl ether, the method comprising:
providing heat to a reboiler of a distillation column of an alkyl tert-butyl ether production unit from a flue gas emanating from a unit carrying out a catalyst regeneration process.

2. The method of claim 1, wherein the distillation column includes a non-reactive distillation column, and/or a reactive distillation column.

3. A method of producing an alkyl tert-butyl ether, the method comprising:
flowing a flue gas stream generated by regenerating a catalyst of a dehydrogenation unit into an air waste heat boiler;
processing the flue gas stream to produce a cooled flue gas stream;
flowing at least a portion of the cooled flue gas stream into a reboiler of a non-reactive distillation column or a reboiler of a reactive distillation column of an alkyl tert-butyl ether production unit; and
providing heat to the reboiler by using the cooled flue gas stream as a heating medium.

4. The method of claim 3, wherein the alkyl tert-butyl ether includes methyl tert-butyl ether (MTBE) and/or ethyl tert-butyl ether (ETBE).

5. The method of claim 3, wherein the unit carrying out the catalyst regeneration process includes an isobutane dehydrogenation unit.

6. The method of claim 5, wherein the isobutane dehydrogenation unit is configured to produce isobutylene as feedstock for MTBE or ETBE synthesis.

7. The method of claim 3, further comprising flowing at least a portion of the cooled flue gas stream into a stack for the air waste heat boiler.

8. The method of claim 7, wherein, by providing heat to the reboiler, the cooled flue gas is further cooled to form an exhaust flue gas flowed from the reboiler to the stack for the air waste heat boiler.

9. The method of claim 6, wherein a tapping device is installed between an outlet of the air waste heat boiler and an inlet of the stack for the air waste heat reboiler, for splitting at least a portion of cooled flue gas stream that is flowed into the reboiler.

10. The method of claim 9, wherein the tapping device includes a valve, a baffle plate, or a damper.

11. (canceled)

12. The method of claim 3, wherein the cooled flue gas stream is flowed through the reboiler by a blower.

13. The method of claim 3, wherein the flue gas stream is at a temperature in a range of 540 to 640° C., and the cooled flue gas stream is at a temperature of 210 to 230° C.

14. The method of claim 3, wherein the flue gas stream comprises 1 to 15 mol. % oxygen gas, 70 to 77 mol. % nitrogen gas, 4 to 6 mol. % CO₂ gas, 2 to 8 mol. % water vapor.

15. The method of claim 3, wherein the regenerating gas can include at least a portion of hot gas from a waste air vent of an MTBE production unit.

16. The method of claim 3, wherein alkyl ether purification column and the reactive distillation column each comprise (1) a flue gas driven reboiler configured to use the cooled flue gas stream as a heating medium and (2) a steam driven reboiler configured to use steam as a heating medium.

17. The method of claim 4, wherein the cooled flue gas stream is flowed through the reboiler by a blower.

18. The method of claim 4, wherein the flue gas stream is at a temperature in a range of 540 to 640° C., and the cooled flue gas stream is at a temperature of 210 to 230° C.

19. The method of claim 4, wherein the flue gas stream comprises 1 to 15 mol. % oxygen gas, 70 to 77 mol. % nitrogen gas, 4 to 6 mol. % CO₂ gas, 2 to 8 mol. % water vapor.

20. The method of claim 4, wherein the regenerating gas can include at least a portion of hot gas from a waste air vent of an MTBE production unit.
